(19) United States
(12) Patent Application Publication
(10) Pub. No.: US 2012/0099773 A1
(43) Pub. Date: Apr. 26, 2012

(54) Method to Achieve Frame Rate or Resolution in Diagnostic Ultrasound

(75) Inventors: Menachem Halmann, Wauwatosa, WI (US); Mohan Krishna Kommu Chinni Subbaraya, Bangalore (IN)

(73) Assignee: GENERAL ELECTRIC COMPANY, Schenectady, NY (US)

(21) Appl. No.: 12/908,566

(22) Filed: Oct. 20, 2010

(51) Int. Cl. G06K 9/00; A61B 8/14

(52) U.S. Cl. 382/131; 600/440

(57) ABSTRACT

Systems and methods for toggling between a high frame rate setting and a high resolution setting while acquiring image data are provided. A method can include: receiving an indication that a high frame rate setting or a high resolution setting should be used to acquire image data, retrieving scan parameters that are associated with the indicated setting from real-time accessible and rewriteable memory, and using the scan parameters to acquire image data using an image acquisition device. An indication to toggle between settings can be triggered manually by an operator of the image acquisition device or automatically based on acquired image data. When successive sets of image data indicate image data is changing at least a minimum amount, a high frame rate setting can be automatically selected. When successive image frames indicate image data is not changing at least a minimum amount, a high resolution setting can be automatically selected.

### RELATED APPLICATIONS

[Not Applicable]

### FEDERALLY SPONSORED RESEARCH OR DEVELOPMENT

[Not Applicable]

### MICROFICHE/COPYRIGHT REFERENCE

[Not Applicable]

### BACKGROUND OF THE INVENTION

Embodiments of the present technology generally relate to two-dimensional (“2D”) and three dimensional (“3D”) imaging. Certain embodiments provide for toggling between a high frame rate setting and a high resolution setting during image data acquisition.

When acquiring imaging data, for example, using an ultrasound imaging system, it is sometimes preferable to obtain images using a high frame rate setting that has a high frequency (i.e., rate) at which unique consecutive images (i.e., frames) are acquired. It is also sometimes preferable to obtain images using a high resolution setting that retains a high level of image detail. However, such settings generally require different image acquisition parameters in order to acquire images at the high frame rate or the high resolution.

Some present image acquisition systems allow for manual toggling between a high frame rate setting and a high resolution setting. However, such systems generally experience a time lag of about 0.5 seconds or more each time they are switched between a high frame rate setting and a high resolution setting. This time lag is noticeable to the end user. However, in clinical settings, it may be preferable to switch between the two settings without noticeable delay.

Thus, there is a need for improved systems and methods that provide for toggling between a high frame rate setting and a high resolution setting during image data acquisition.

### BRIEF SUMMARY OF THE INVENTION

Certain embodiments of the present technology provide systems, methods and non-transitory computer readable mediums encoded with instructions for toggling between a high frame rate setting and a high resolution setting while acquiring image data.

In certain embodiments, for example, a method for toggling between a high frame rate setting and a high resolution setting while acquiring image data includes: receiving an indication that a high frame rate setting or a high resolution setting should be used to acquire image data using an image acquisition device; retrieving scan parameters from a real-time accessible and rewriteable memory location that is operably connected to the image acquisition device, the scan parameters associated with the indicated high frame rate setting or high resolution setting; and using the retrieved scan parameters to acquire image data using the image acquisition device.

In certain embodiments, for example, the indication that the high frame rate setting or the high resolution setting should be used to acquire image data is triggered automatically based on acquired image data.

In certain embodiments, for example, a method further includes: using the image acquisition device to acquire a plurality of successive sets of image data; determining an amount of change between the successive sets of image data; determining whether the amount of change between the successive sets of image data is at least a threshold amount of change; determining whether the amount of change has been at least the threshold amount for a predetermined number of successive sets of image data; if the amount of change has been at least the threshold amount for the predetermined number of successive sets of image data, automatically indicating that the high frame rate setting for image acquisition should be used; and if the amount of change has not been at least the threshold amount for the predetermined number of successive sets of image data, automatically indicating that the high resolution setting for image acquisition should be used.

In certain embodiments, for example, the amount of change between the successive sets of image data is determined using one or more of the following: histograms of the successive sets of image data, mean values of the successive sets of image data, standard deviations of the successive sets of image data, a cross-correlation of the successive sets of image data, and a sum of the absolute differences between of the successive sets of image data.

In certain embodiments, for example, the indication that the high frame rate setting or the high resolution setting should be used to acquire image data is triggered manually by an operator of the image acquisition device.

In certain embodiments, for example, the image acquisition device includes a transducer comprising a manual switch, and the method further includes using the manual switch to toggle between the high frame rate setting and the high resolution setting.

In certain embodiments, for example, a method further includes: storing the high frame rate setting and the high resolution setting in the real-time accessible and rewriteable memory location when the image acquisition device is activated.

In certain embodiments, for example, a system for acquiring image data includes: a computer processor configured to receive an indication that a high frame rate setting or a high resolution setting should be used to acquire image data using an image acquisition device, the computer processor configured to retrieve scan parameters from a real-time accessible and rewriteable memory location that is operably connected to the computer processor, the scan parameters associated with the indicated high frame rate setting or high resolution setting; and the computer processor configured to use the retrieved scan parameters to acquire image data using the image acquisition device.

In certain embodiments, for example, the computer processor is configured to automatically indicate whether the high frame rate setting or the high resolution setting should be used to acquire image data.

In certain embodiments, for example, the image acquisition device is configured to acquire a plurality of successive sets of image data, wherein the computer processor is configured to determine an amount of change between the successive sets of image data, wherein the computer processor is configured to determine whether the amount of change between the successive sets of image data is at least a threshold amount of change, wherein the computer processor is configured to determine whether the amount of change has been at least the threshold amount for a predetermined number of successive sets of image data, wherein the computer processor is configured such that, if the amount of change has been at least the threshold amount for the predetermined number of successive sets of image data, the computer processor automatically indicates that the high frame rate setting for image acquisition should be used; and wherein the computer processor is configured such that, if the amount of change has not been at least the threshold amount for the predetermined number of successive sets of image data, the computer processor automatically indicates that the high resolution setting for image acquisition should be used.

In certain embodiments, for example, the computer processor is configured to determine amount of change between the successive sets of image data using one or more of the following: histograms of the successive sets of image data, mean values of the successive sets of image data, standard deviations of the successive sets of image data, a cross-correlation of the successive sets of image data, and a sum of the absolute differences between the successive sets of image data.

In certain embodiments, for example, the computer processor is configured to indicate that the high frame rate setting or the high resolution setting should be used to acquire image data based on a manually triggered input by an operator of the image acquisition device.

In certain embodiments, for example, the image acquisition device includes a transducer comprising a manual switch, wherein triggering the manual switch indicates to the computer processor to toggle between the high frame rate setting and the high resolution setting.

In certain embodiments, for example, the image acquisition device is an ultrasound imaging system.

In certain embodiments, for example, a non-transitory computer-readable storage medium encoded with a set of instructions for execution on a processing device and associated processing logic, wherein the set of instructions includes: a first routine configured to receive an indication that a high frame rate setting or a high resolution setting should be used to acquire image data using an image acquisition device; a second routine configured to retrieve scan parameters from a real-time accessible and rewriteable memory location that is operably connected to the image acquisition device, the scan parameters associated with the indicated high frame rate setting or high resolution setting; and a third routine configured to use the retrieved scan parameters to acquire image data using the image acquisition device.

In certain embodiments, for example, the indication that the high frame rate setting or the high resolution setting should be used to acquire image data is triggered automatically based on acquired image data.

In certain embodiments, for example, the medium and instructions further includes: a fourth routine configured to use the image acquisition device to acquire a plurality of successive sets of image data; a fifth routine configured to determine an amount of change between the successive sets of image data; a sixth routine configured to determine whether the amount of change between the successive sets of image data is at least a threshold amount of change; a seventh routine configured to determine whether the amount of change has been at least the threshold amount for a predetermined number of successive sets of image data; an eighth routine configured such that, if the amount of change has been at least the threshold amount for the predetermined number of successive sets of image data, the eighth routine automatically indicates that the high frame rate setting for image acquisition should be used; and a ninth routine configured such that, if the amount of change has not been at least the threshold amount for the predetermined number of successive sets of image data, the ninth routine automatically indicates that the high resolution setting for image acquisition should be used.

In certain embodiments, for example, the amount of change between the first set of image data and the second set of image data is determined using one or more of the following: histograms of the successive sets of image data, mean values of the successive sets of image data, standard deviations of the successive sets of image data, a cross-correlation of the successive sets of image data, and a sum of the absolute differences between the successive sets of image data.

In certain embodiments, for example, the indication that the high frame rate setting or the high resolution setting should be used to acquire image data is triggered manually by an operator of the image acquisition device.

In certain embodiments, for example, the image acquisition device includes a transducer comprising a manual switch, and the medium and instructions further includes a fourth routine configured to toggle between the high frame rate setting and the high resolution setting when the manual switch is triggered.

### BRIEF DESCRIPTION OF THE DRAWINGS

FIG. 1 illustrates a block diagram of an ultrasound imaging system used in accordance with an embodiment of the present technology.

FIG. 2 illustrates a block diagram of a system for toggling between a high frame rate setting and a high resolution setting used in accordance with an embodiment of the present technology.

FIG. 3 illustrates a method for automatically selecting a high frame rate setting or a high resolution setting during image acquisition used in accordance with an embodiment of the present technology.

The foregoing summary, as well as the following detailed description of embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF CERTAIN EMBODIMENTS

Embodiments of the present technology generally relate to two-dimensional (“2D”) and three dimensional (“3D”) imaging. Certain embodiments provide for toggling between a high frame rate setting and a high resolution setting during data acquisition.

It has been found that existing systems that provide for manual toggling between a high frame rate setting and a high resolution setting generally experience a time lag of about 0.5 seconds or more upon switching, which time lag is noticeable to a user. It has been found that the time lag can sometimes be attributed to calculating scan parameters upon switching. It has also been found that the time lag can sometimes be attributed to loading scan parameters from memory locations that are not real-time accessible, such as from a hard drive, for example.

It has been found that maintaining scan parameters for both a high frame rate setting and a high resolution setting in memory locations that are real-time accessible and rewriteable, such as RAM and/or processor registers, for example, can allow for toggling between the two settings in less than about 0.1 seconds, which is not noticeable to a user. In other words, maintaining scan parameters for both a high frame rate setting and a high resolution setting in memory locations that are real-time accessible and rewriteable can allow for toggling between the two settings during data acquisition without any perceived delay.

Certain embodiments provide for manual and/or automatic switching between a high frame rate setting and a high resolution setting. In certain embodiments, a user interface of an imaging system can be used to select whether manual switching, automatic switching or both are activated.

In certain manual switching embodiments, a user can direct the imaging system to toggle between the high frame rate setting and the high resolution setting using a switch accessible on a user interface of the imaging system and/or using a switch accessible on a transducer of the imaging system. In certain embodiments, the manual switch can comprise a button, knob, switch, trackball, slider, menu option displayed on a graphical user interface, and/or a keystroke, for example.

In automatic switching embodiments, either a high resolution setting or a high frame rate setting can be automatically selected based on thresholds of change between successive data sets (e.g., successive 2D image frames and/or successive 3D volumes) during data acquisition. For example, in certain embodiments, the imaging system can switch to the high resolution setting if a threshold number of successive data sets do not exhibit a threshold amount of change from the immediately preceding data sets. In certain embodiments, the threshold number of successive data sets can be between 1 and about 5 data sets, for example, and the threshold amount of change between successive data sets can vary depending on the manner in which change between data sets is being calculated.

In certain embodiments, for example, the imaging system can switch to the high frame rate setting if a threshold number of successive data sets exhibit at least a threshold amount of change from the immediately preceding data sets. In certain embodiments, the threshold number of successive data sets can be between 1 and about 5 data sets, for example, and the threshold amount of change between successive data sets can vary depending on the manner in which change between data sets is being calculated.

Change between data sets can be calculated in various ways. For example, in certain embodiments, histograms for successive data sets can be compared to determine whether a threshold amount of change has occurred between successive data sets. A histogram provides the distribution of gray scales across the data set by counting the number of pixels that have the same gray level. For example, gray level 0 (black) may have 1000 pixels and gray level 1 (almost black) may have 510 pixels. In certain embodiments, an algorithm provided as computer readable code, for example, can determine the gray scale change between successive images by determining the difference in the number of pixels at each gray level (e.g., gray level 0=black, gray level 1=almost black, gray level 254=almost white, gray level 255=white). Change in number of pixels at each gray level can be provided as a percent change for that gray level. The percent change can be averaged for all gray levels to provide an overall change. If the overall change is less than a pre-determined threshold, such as 3%, for example, the data sets can be determined to be constant enough to warrant a high-resolution setting. If the overall change is at or above the pre-determined threshold, the data sets can be determined to be changing enough to warrant a high frame rate setting.

In certain embodiments, for example, mean gray level values and standard deviations for pixels in successive data sets can be used to determine whether a threshold amount of change has occurred between a first data set and the next successive data set. For example, for each pixel represented in multiple data sets, or for a subset of pixels represented in multiple data sets, a mean (average) gray level value and a standard deviation from the mean gray level value can be calculated.

For example, if eight successive data sets are used to generate a mean gray level value and standard deviation, and the pixel has gray level values 2, 4, 4, 4, 5, 5, 7 and 9 in the eight successive data sets, the mean (average) gray level value=(2+4+4+4+5+5+7+9)/8=5. To calculate the standard deviation for the eight values of the pixel, first the difference of each pixel value from the mean gray level value is taken and squared as follows: (2−5)̂2=(−3)̂2=9; (5−5)̂2=0̂2=0; (4−5)̂2=(−1)̂2=1; (5−5)̂2=0̂2=0; (7−5)̂2=2̂2=4; (9−5)̂2=4̂2=16. Next, those numbers are averaged and the square root is taken as follows: sqrt([9+1+1+1+0+0+4+16]/8)=2. Thus, two is the standard deviation for the eight successive data sets.

In certain embodiments, an algorithm provided as computer readable code, for example, can determine a mean gray level value and a standard deviation value for each pixel represented in multiple successive data sets, or for a subset of pixels represented in multiple successive data sets. The standard deviation values can be averaged to determine an average gray level standard deviation. The average gray level standard deviation can be provided as a percent of the total number of gray levels. For example, an average gray level standard deviation of 12 gray levels for a gray scale of 256 gray levels corresponds with a change in gray level of about 5%. If the percent is less than a pre-determined threshold, such as 5%, for example, the data sets can be determined to be constant enough to warrant a high-resolution setting. If the overall change is at or above the pre-determined threshold, the data sets can be determined to be changing enough to warrant a high frame rate setting.

In certain embodiments, cross correlation and/or absolute difference between successive data sets can be used to determine change in a manner similar to standard deviation. In certain embodiments, only the change between successive images is written to a buffer to determine whether a threshold amount of change has occurred between successive data sets.

In certain embodiments, utilizing thresholds as described above can avoid excessive switching between a high frame rate setting and a high resolution setting. In certain embodiments, a user interface of an imaging system can be used to change the threshold number of successive data sets and/or the threshold amount of change between successive data sets, such that a user can customize the automatic switching function.

In certain embodiments, a user interface of an imaging system can indicate whether a high frame rate setting or a high resolution setting is being used.

While certain embodiments described below are discussed in connection with ultrasound imaging systems that acquire images as 2D frames, the inventions disclosed herein are not limited to such applications. The inventions herein can be used in connection with any image acquisition application where it is desirable to toggle between a high frame rate setting and a high resolution setting. For example, certain embodiments can be used in connection with 3D imaging systems and probes that acquire imaging data sets for 3D volumes.

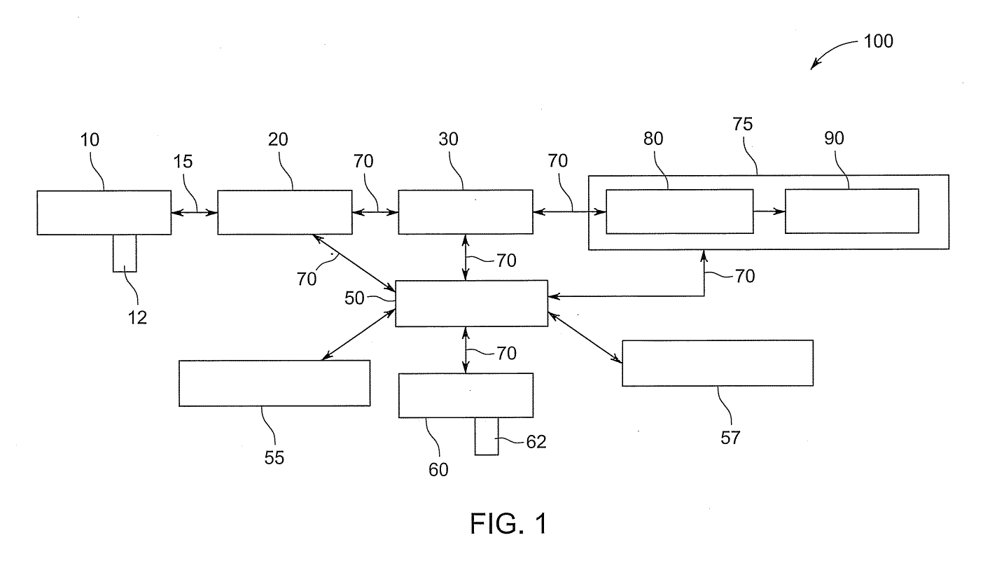

FIG. 1 illustrates a block diagram of an ultrasound imaging system 100 used in accordance with an embodiment of the present technology. The system 100 includes a transducer 10 with manual switch 12, a front-end 20, an imaging mode processor 30, a user interface 60 with manual switch 62, a control processor 50, a data storage 55, real-time accessible and rewriteable memory 57, and a display 75. In certain embodiments, the imaging mode processor 30 and the control processor 50 may be part of a back-end system.

Transducer 10 and front-end 20 can be used together to create a beam pattern that is used to create an image. Transducer 10 can be used to transmit ultrasound waves into a subject by converting electrical analog signals to ultrasonic energy. The transducer 10 can also be used to detect ultrasound waves that are backscattered from the subject by converting ultrasonic energy to analog electrical signals. In certain embodiments, the transducer 10 can be a linear array or a phased array. Transducer 10 includes manual switch 12 that can be used to manually toggle between a high frame rate setting and a high resolution setting during image acquisition. In certain embodiments, manual switch 12 can comprise a button, knob, switch, trackball, and/or a slider, for example. In certain embodiments, manual switch 12 can be operably connected to an electronic switch, such as a wired or wireless switch integrated with the transducer's electronics. The electronic switch can signal to processor 50 that a high frame rate setting or a high resolution setting is to be used during image acquisition.

The front-end 20 can include a receiver, a transmitter and/or a beamformer. The front-end 20 can be used to create transmitted waveforms, beam patterns, receiver filtering techniques, and demodulation schemes that can be used for various imaging modes. The front-end 20 can interface with the transducer 10 via an analog interface 15. The front-end 20 can interface with the imaging mode processor 30 and the control processor 50 via a digital bus 70. The digital bus 70 can include several digital sub-buses. The digital sub-buses can have separate configurations and provide digital data interfaces to various parts of the ultrasound imaging system 100.

Once a beam pattern has been focused, the beam pattern can be output from the front-end 20 to the imaging mode processor 30 in the form of digital signal data. The imaging mode processor 30 can process the received digital signal data to produce estimated parameter values. The imaging mode processor 30 can pass the estimated parameter values to a control processor 50 over the digital bus 70. The imaging mode processor 30 can also pass the estimated parameter values to the display 75 via the digital bus 70.

The display 75 can include a display processor 80 and a monitor 90. The display processor 80 can accept digital parameter values from the imaging mode processor 30 and the control processor 50. The display processor 80 can perform scan-conversion functions, color mapping functions, and tissue/flow arbitration functions, for example. The display processor 80 can process map and format the digital data for display, convert the digital display data to analog display signals, and pass the analog display signals to the monitor 90. The monitor 90 can accept the analog display signals from the display processor 80 and display the resulting image. An operator may view the image on the monitor 90.

The control processor 50 is the central processor of the ultrasound imaging system 100, and can comprise any processing device capable of executing computer-readable code. The control processor 50 can interface with other components of the ultrasound imaging system 100 using the digital bus 70. The control processor 50 can execute various data algorithms and functions for various imaging and diagnostic modes. Digital data and commands can be transmitted and received between the control processor 50 and other components of the ultrasound imaging system 100. In certain embodiments, functions performed by the control processor 50 can be performed by multiple processors and/or can be integrated into the imaging mode processor 30 and/or the display processor 80. In another embodiment, the functions of the processors 30, 50, and 80 can be integrated into a single personal computer (“PC”) backend.

Real-time accessible and rewriteable memory 57 can comprise any computer memory location from which data can be retrieved in substantially real-time (e.g., without noticeable delay to an end user), and to which data can be written. In certain embodiments, real-time accessible and rewriteable memory 57 can comprise RAM and/or processor registers, for example, and can allow for toggling between stored settings for high frame rate and high resolution in less than about 0.1 seconds, which is not noticeable to a user. RAM can be any tangible, non-transitory computer-readable medium that is readable by processor 50, and allows stored data to be accessed in any order (i.e., at random) regardless of the physical location of the data, and whether or not the data is related to a previous piece of data. Non-RAM computer storage includes mediums that rely on physical movement of the recording medium in order to retrieve data, such as magnetic discs and optical discs, for example.

Data storage 55 can be any tangible, non-transitory computer-readable medium that is readable by processor 50, whether local, remote, connected by wires and/or connected wirelessly. For example, storage medium 55 can include a computer hard drive, a server, a CD, a DVD, a USB thumb drive, and/or any other type of tangible memory capable of storing one or more computer instructions. The sets of instructions can include one or more routines capable of being run or performed by processor 50.

The user interface 60 can allow user commands to be input by the operator to the ultrasound imaging system 100 through the control processor 50. The user interface 60 can include a keyboard, mouse, switches, knobs, buttons, track ball, and/or on screen menus, for example. User interface 60 includes manual switch 62 that can be used to manually switch between a high frame rate setting and a high resolution setting during image acquisition. In certain embodiments, manual switch 62 can comprise a button, knob, switch, trackball, slider, menu option displayed on a graphical user interface, and/or a keystroke, for example.

Ultrasound imaging system 100 is configured to maintain various sets of scan parameters in real-time accessible and rewriteable memory 57. For example, in certain embodiments, real-time accessible and rewriteable memory 57 can include a first set of scan parameters for a high frame rate setting and a second set of scan parameters for a high resolution setting. In an embodiment, a set of scan parameters includes parameters for: line density, number of focal points, frame averaging, speckle reduction filter, compounding, and scan width. In an embodiment, a set of scan parameters for a high frame rate setting can provide low line density, such as a line density of 128 lines per frame, for example, one focal point, no frame averaging, no speckle reduction filter, no compounding, and low scan width. In certain embodiments, such high frame rate scan parameters can provide for a frame rate of at least about 50 Hz. In an embodiment, a set of scan parameters for a high resolution setting can provide high line density, such as a line density of 256 lines per frame, for example, a plurality of focal points, substantially optimal frame averaging, such as frame averaging of 3, for example, speckle reduction filter on, compounding on, and high scan width.

Processor 50 is configured to obtain scan parameters from real-time accessible and rewriteable memory 57 that correspond to the current setting, high frame rate or high resolution. The retrieved scan parameters are used by ultrasound imaging system 100 to define beam transmission control, receive beamforming control, and scan conversion control.

In certain embodiments, the scan setting (high frame rate or high resolution) can be manually toggled using manual switch 12 on transducer 10 and/or using manual switch 62 on user interface 60.

In certain embodiments, the scan setting (high frame rate or high resolution) can be automatically toggled depending on whether the transducer 10 is moving or remains still. To determine whether the transducer 10 is moving or remains substantially still, data from successive image frames can be compared to detect change (or lack of change) between the frames.

In certain embodiments, raw image data collected by transducer 10 for successive frames is compared to determine change (or lack of change) between the frames. As described above, the amount of change between frames can be determined in various manners, including: by comparing histograms, mean values, and/or standard deviations of successive frames, by cross-correlating complete images in successive frames, and/or by summing the absolute differences between images in successive frames.

In certain embodiments, threshold values for the amount of change between successive frames can be set such that less than the threshold amount of change indicates transducer 10 remains substantially still and a high resolution setting is preferable, and more than the threshold amount of change indicates transducer 10 is moving and a high frame rate setting is preferable.

In certain embodiments, data from a threshold number of frames can be considered prior to automatically toggling between a high frame rate setting and a high resolution setting. For example, in certain embodiments, if the threshold amount of change is exceeded in each of three successive frames, the system can automatically toggle from a high resolution setting to a high frame rate setting. Conversely, if the threshold amount of change is not exceeded in each of three successive frames, the system can automatically toggle from a high frame rate setting to a high resolution setting. In certain embodiments, the threshold number of frames to consider prior to automatically toggling between a high frame rate setting and a high resolution setting can be any number of frames, such as 1, 2, 3, 4, 5, 6, 7, 8, 9 or 10 frames, for example.

In certain embodiments, utilizing thresholds as described above can avoid excessive switching between a high frame rate setting and a high resolution setting because the system will not toggle the setting (i.e., the present setting is maintained) until the threshold number of successive frames are either: (1) above the threshold amount of change, or (2) at or below the threshold amount of change. In certain embodiments, user interface 60 can be used to change the threshold number of successive frames and/or the threshold amount of change between successive frames, such that a user can customize the automatic switching function. Such a customized setting can be saved in data storage 55 such that each time the user activates a transducer, the user's toggle settings are retrieved and applied by processor 50.

In operation, ultrasound imaging system 100 can acquire sets of image data as frames using scan parameters associated with a high frame rate setting or a high resolution setting. The scan parameters can be retrieved from real-time accessible and rewriteable memory 57, where both sets of scan parameters are maintained. When there is an indication that the setting should be toggled (from a high frame rate setting to a high resolution setting or vice versa), acquisition of image data for the frame that is presently being acquired can be completed using the present setting (and associated scan parameters), and image data for the next frame can be acquired using the other setting (and associated scan parameters). The indication to toggle between the two settings can be the result of a manual switch being triggered by an operator of ultrasound imaging system 100 and/or can be automatically triggered based on movement or non-movement of transducer 10, as described above. Also, even if the transducer is not moving, movement of the target, such as anatomy including a beating heart, for example, may cause enough change in the acquired image data to trigger automatic selection of a high frame rate setting.

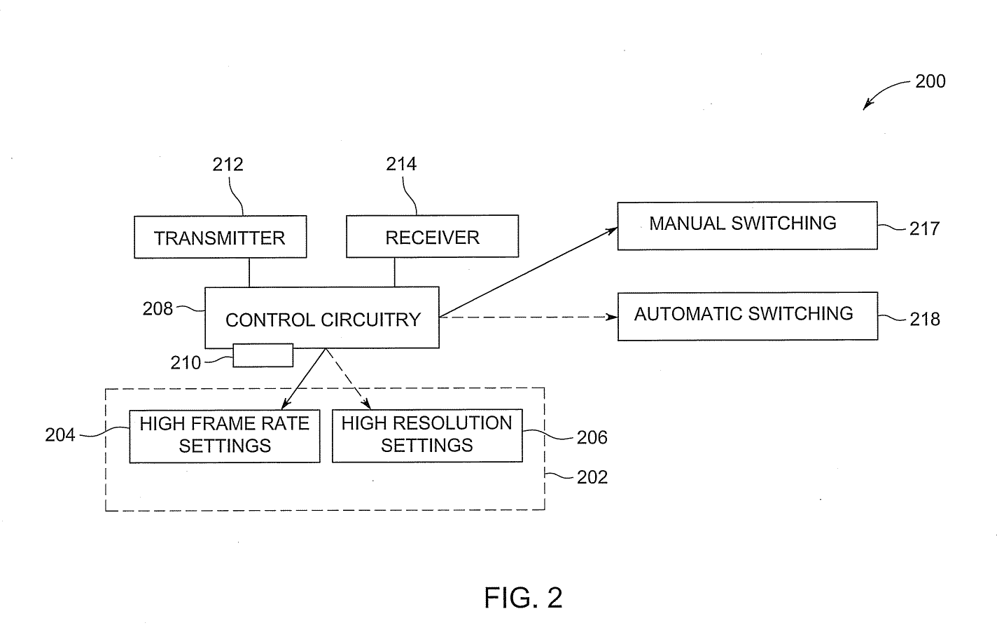

FIG. 2 illustrates a block diagram of a system 200 for toggling between a high frame rate setting and a high resolution setting used in accordance with an embodiment of the present technology. The system includes real-time accessible and rewriteable memory 202 operably connected to control circuitry 208, which is operably connected to a transmitter 212 and receiver 214. Transmitter 212 and receiver 214 are part of an image acquisition device configured to obtain successive sets of image data for a target, such as human anatomy, for example. The image acquisition device can obtain image data using high frame rate settings 204 or high resolution settings 206.

High frame rate settings 204 and high resolution settings 206 are stored in real-time accessible and rewriteable memory 202 when the image acquisition device, such as a transducer comprising transmitter 212 and receiver 214, for example, is activated. In certain embodiments, once the settings 204, 206 are stored in real-time accessible and rewriteable memory 202, they need not be stored there again, and can be retrieved from real-time accessible and rewriteable memory 202 any time during operation of the image acquisition device in substantially real time (e.g. without noticeable delay to a user of the image acquisition device). In operation, the system 200 can toggle between the settings 204, 206 based on an indication to switch received from control circuitry 208.

Control circuitry includes switch 210 that can be used to provide the indication to toggle between settings 204 and 206. Control circuitry 208 is configured to provide for switching based on a manual indication and/or an automatic indication in a manner similar to that described above. In certain embodiments, a user of the image acquisition device can select whether switching will be controlled manually 217, automatically 218, or both manually 217 and automatically 218.

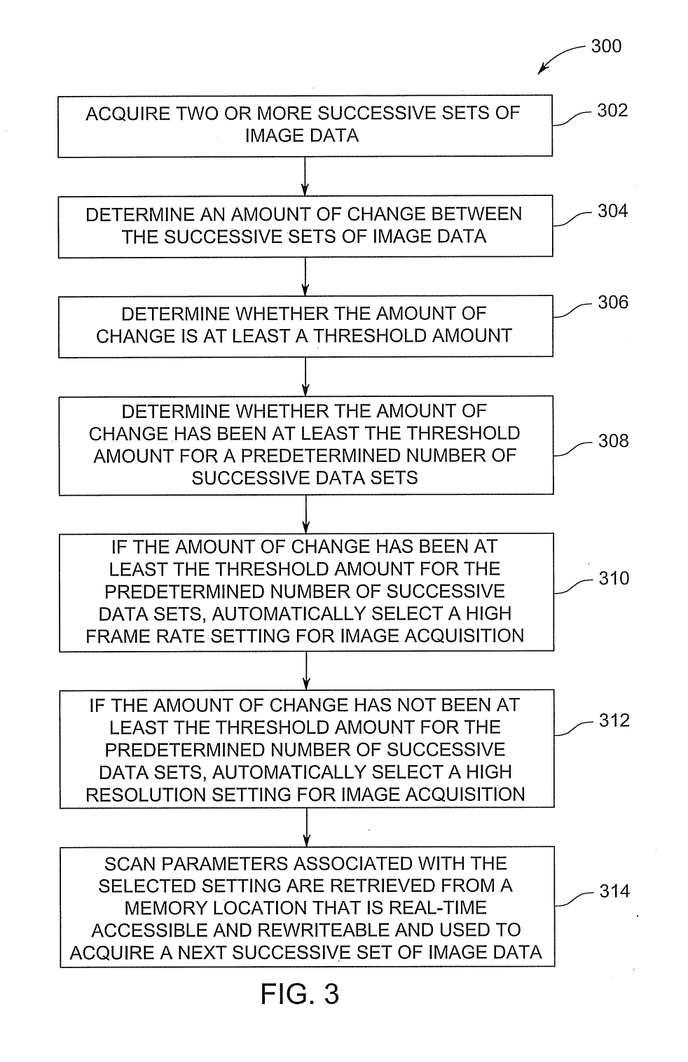

FIG. 3 illustrates a method 300 for automatically selecting a high frame rate setting or a high resolution setting during image acquisition used in accordance with an embodiment of the present technology. The method can be applied by employing the techniques and systems described herein.

At 302, two or more successive sets of image data are acquired. For example, in an embodiment, an ultrasound imaging system can acquire two or more successive sets of image data.

At 304, an amount of change between successive sets of image data is determined. For example, in an embodiment, an ultrasound imaging system can use a processor to determine an amount of change between successive sets of image data by comparing histograms, mean values, and/or standard deviations of sets of image data, by cross-correlating complete images and/or by summing the absolute differences between images.

At 306, whether the amount of change is at least a threshold amount is determined. For example, in an embodiment, an ultrasound imaging system can use a processor to determine whether the amount of change between the successive sets of image data is at least a threshold amount of change.

At 308, whether the amount of change has been at least the threshold amount for a predetermined number of successive sets of image data is determined. For example, in an embodiment, an ultrasound imaging system can use a processor to determine whether the amount of change between successive sets of image data has been at least the threshold amount for a predetermined number of sets of image data, such as for 1, 2, 3, 4, 5, 6, 7, 8, 9, or 10 consecutive sets of image data, for example.

At 310, if the amount of change has been at least the threshold amount for the predetermined number of successive sets of image data, a high frame rate setting for image acquisition is automatically selected. For example, in an embodiment, an ultrasound imaging system can use a processor to automatically select a high frame rate setting for image acquisition if the amount of change has been at least the threshold amount for the predetermined number of successive sets of image data.

At 312, if the amount of change has not been at least the threshold amount for the predetermined number of successive sets of image data, a high resolution setting for image acquisition is automatically selected. For example, in an embodiment, an ultrasound imaging system can use a processor to automatically select a high resolution setting for image acquisition if the amount of change has not been at least the threshold amount for the predetermined number of successive sets of image data.

At 314, scan parameters associated with the selected setting are retrieved from real-time accessible and rewriteable memory and used to acquire a next successive set of image data. For example, in an embodiment, an ultrasound imaging system can use a processor to retrieve scan parameters associated with the selected setting from RAM and/or processor registers and use the retrieved scan parameters to acquire a next successive set of image data.

Certain embodiments of the present invention may omit one or more of the steps and/or perform the steps in a different order than the order listed in connection with FIG. 3. For example, some steps may not be performed in certain embodiments of the present invention. As a further example, certain steps may be performed in a different temporal order, including simultaneously, than listed above.

One or more of the steps of the method 300 may be implemented alone or in combination in hardware, firmware, and/or as a set of instructions in software, for example. Certain embodiments may employ the ultrasound imaging system 100 described herein including processor 50 to achieve the method steps. Certain embodiments may employ the system 200 described herein including control circuitry 208 to achieve the method steps.

Certain embodiments may be provided as a set of instructions residing on a tangible, non-transitory computer-readable medium, such as a memory, hard disk, DVD, or CD, for execution on a general purpose computer or other processing device. For example, certain embodiments provide a non-transitory computer-readable storage medium encoded with a set of instructions for execution on a processing device and associated processing logic, wherein the set of instructions includes a routine(s) configured to provide the functions described in connection with the systems and methods described herein.

Applying systems and techniques described herein, can provide a technical effect of automatically or manually toggling between a high frame rate setting and a high resolution setting during image acquisition without noticeable delay to the operator of an imaging system.

Certain image data acquired, analyzed and displayed in connection with techniques described herein represent human anatomy. In other words, outputting a visual display based on such data comprises a transformation of underlying subject matter (such as an article or materials) to a different state.

While the invention has been described with reference to embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

What is claimed is:

1. A method for toggling between a high frame rate setting and a high resolution setting while acquiring image data comprising:
receiving an indication that a high frame rate setting or a high resolution setting should be used to acquire image data using an image acquisition device;
retrieving scan parameters from a real-time accessible and rewriteable memory location that is operably connected to the image acquisition device, the scan parameters associated with the indicated high frame rate setting or high resolution setting; and
using the retrieved scan parameters to acquire image data using the image acquisition device.

2. The method of claim 1, wherein the indication that the high frame rate setting or the high resolution setting should be used to acquire image data is triggered automatically based on acquired image data.

3. The method of claim 2, further comprising:
using the image acquisition device to acquire a plurality of successive sets of image data;
determining an amount of change between the successive sets of image data;
determining whether the amount of change between the successive sets of image data is at least a threshold amount of change;
determining whether the amount of change has been at least the threshold amount for a predetermined number of successive sets of image data;
if the amount of change has been at least the threshold amount for the predetermined number of successive sets of image data, automatically indicating that the high frame rate setting for image acquisition should be used; and
if the amount of change has not been at least the threshold amount for the predetermined number of successive sets of image data, automatically indicating that the high resolution setting for image acquisition should be used.

4. The method of claim 3, wherein the amount of change between the successive sets of image data is determined using one or more of the following:
histograms of the successive sets of image data,
mean values of the successive sets of image data,
standard deviations of the successive sets of image data,
a cross-correlation of the successive sets of image data, and
a sum of the absolute differences between of the successive sets of image data.

5. The method of claim 1, wherein the indication that the high frame rate setting or the high resolution setting should be used to acquire image data is triggered manually by an operator of the image acquisition device.

6. The method of claim 5, wherein the image acquisition device includes a transducer comprising a manual switch, and the method further includes using the manual switch to toggle between the high frame rate setting and the high resolution setting.

7. The method of claim 1, further comprising storing the high frame rate setting and the high resolution setting in the real-time accessible and rewriteable memory location when the image acquisition device is activated.

8. A system for acquiring image data comprising:
a computer processor configured to receive an indication that a high frame rate setting or a high resolution setting should be used to acquire image data using an image acquisition device,
the computer processor configured to retrieve scan parameters from a real-time accessible and rewriteable memory location that is operably connected to the computer processor, the scan parameters associated with the indicated high frame rate setting or high resolution setting; and
the computer processor configured to use the retrieved scan parameters to acquire image data using the image acquisition device.

9. The system of claim 8, wherein the computer processor is configured to automatically indicate whether the high frame rate setting or the high resolution setting should be used to acquire image data.

10. The system of claim 9, wherein the image acquisition device is configured to acquire a plurality of successive sets of image data,
wherein the computer processor is configured to determine an amount of change between the successive sets of image data,
wherein the computer processor is configured to determine whether the amount of change between the successive sets of image data is at least a threshold amount of change,
wherein the computer processor is configured to determine whether the amount of change has been at least the threshold amount for a predetermined number of successive sets of image data,
wherein the computer processor is configured such that, if the amount of change has been at least the threshold amount for the predetermined number of successive sets of image data, the computer processor automatically indicates that the high frame rate setting for image acquisition should be used; and
wherein the computer processor is configured such that, if the amount of change has not been at least the threshold amount for the predetermined number of successive sets of image data, the computer processor automatically indicates that the high resolution setting for image acquisition should be used.

11. The system of claim 10, wherein the computer processor is configured to determine amount of change between the successive sets of image data using one or more of the following:
histograms of the successive sets of image data,
mean values of the successive sets of image data,
standard deviations of the successive sets of image data,
a cross-correlation of the successive sets of image data, and
a sum of the absolute differences between the successive sets of image data.

12. The system of claim 8, wherein the computer processor is configured to indicate that the high frame rate setting or the high resolution setting should be used to acquire image data based on a manually triggered input by an operator of the image acquisition device.

13. The system of claim 12, wherein the image acquisition device includes a transducer comprising a manual switch, wherein triggering the manual switch indicates to the computer processor to toggle between the high frame rate setting and the high resolution setting.

14. The system of claim 8, wherein the image acquisition device is an ultrasound imaging system.

15. A non-transitory computer-readable storage medium encoded with a set of instructions for execution on a processing device and associated processing logic, wherein the set of instructions includes:
a first routine configured to receive an indication that a high frame rate setting or a high resolution setting should be used to acquire image data using an image acquisition device;
a second routine configured to retrieve scan parameters from a real-time accessible and rewriteable memory location that is operably connected to the image acquisition device, the scan parameters associated with the indicated high frame rate setting or high resolution setting; and
a third routine configured to use the retrieved scan parameters to acquire image data using the image acquisition device.

16. The medium and instructions of claim 15, wherein the indication that the high frame rate setting or the high resolution setting should be used to acquire image data is triggered automatically based on acquired image data.

17. The medium and instructions of claim 16, further comprising:
a fourth routine configured to use the image acquisition device to acquire a plurality of successive sets of image data;
a fifth routine configured to determine an amount of change between the successive sets of image data;
a sixth routine configured to determine whether the amount of change between the successive sets of image data is at least a threshold amount of change;
a seventh routine configured to determine whether the amount of change has been at least the threshold amount for a predetermined number of successive sets of image data;
an eighth routine configured such that, if the amount of change has been at least the threshold amount for the predetermined number of successive sets of image data, the eighth routine automatically indicates that the high frame rate setting for image acquisition should be used; and
a ninth routine configured such that, if the amount of change has not been at least the threshold amount for the predetermined number of successive sets of image data, the ninth routine automatically indicates that the high resolution setting for image acquisition should be used.

18. The medium and instructions of claim 17, wherein the amount of change between the first set of image data and the second set of image data is determined using one or more of the following:
histograms of the successive sets of image data,
mean values of the successive sets of image data,
standard deviations of the successive sets of image data,
a cross-correlation of the successive sets of image data, and
a sum of the absolute differences between the successive sets of image data.

19. The medium and instructions of claim 15, wherein the indication that the high frame rate setting or the high resolution setting should be used to acquire image data is triggered manually by an operator of the image acquisition device.

20. The medium and instructions of claim 19, wherein the image acquisition device includes a transducer comprising a manual switch, and the medium and instructions further includes a fourth routine configured to toggle between the high frame rate setting and the high resolution setting when the manual switch is triggered.
